(19) United States
(12) Patent Application Publication
(10) Pub. No.: US 2026/0346034 A1
(43) Pub. Date: Nov. 5, 2026

(54) TRAINING DEVICE FOR LOWERING INTRAOCULAR PRESSURE

(71) Applicants: Zhuhai ULook MetaBrain Medical Technology Co. Ltd., Zhuhai City (CN); METABRAIN TECHNOLOGY PTE.LTD., SINGAPORE (SG)

(72) Inventors: Wen-Fang LEI, Taoyuan City (TW); Shin-Da LEE, Taichung City (TW); Kai-Po CHANG, Taichung City (TW)

(21) Appl. No.: 19/194,529

(22) Filed: Apr. 30, 2025

(30) Foreign Application Priority Data
Oct. 10, 2024 (CN) 202411408932.X

(51) Int. Cl. A61N 2/00; A61M 21/00; A61M 21/02; A61N 2/02; A61N 5/06; A61N 5/067; A63B 21/005; A63B 21/22; A63B 22/06; A63B 71/06

(52) U.S. Cl. CPC A61N 2/002; A61M 21/02; A61N 2/006; A61N 2/02; A61N 5/0619; A61N 5/067; A63B 21/0054; A63B 21/0055; A63B 21/225; A63B 22/0605; A63B 71/0622; A61M 2021/0027; A61M 2021/005; A61M 2205/054; A61M 2205/057; A61M 2205/50; A61M 2209/088; A63B 2071/0638

(57) ABSTRACT

A training device for lowering intraocular pressure includes a bracket unit, a virtual reality unit, a binaural audio stimulation unit, a transcranial stimulation unit and a control unit; the bracket unit includes top and back side parts and two ears part; the virtual reality unit includes display headset; the binaural audio stimulation unit includes paired speakers, wherein the paired speakers are used for playing binaural beats music to lower intraocular pressure; the transcranial stimulation unit includes two first stimulus mediums, the first stimulus mediums are used for transcranial electrical stimulation or transcranial magnetic stimulation; The training device for lowering intraocular pressure for users provided by the invention can perform various trainings for lowering intraocular pressure in a single course of treatment through the display headset, the audio speaker and the transcranial stimulation medium, so as to synergistically achieve the effect of reducing intraocular pressure of users.

### BACKGROUND OF THE INVENTION

### Field of the Invention

The invention relates to the technical field of intraocular pressure treatment, in particular to a training device for lowering intraocular pressure.

### Description of the Prior Art

Glaucoma is an eye disease that can lead to blindness. It is generally believed that high intraocular pressure is a risk factor for glaucoma. At present, most of the treatment methods use drugs or surgery to reduce intraocular pressure; however, besides drugs and surgery, how to reduce intraocular pressure by non-invasive approaches is also the focus of relevant research at present.

The present invention is, therefore, arisen to obviate or at least mitigate the above-mentioned disadvantages.

### SUMMARY OF THE INVENTION

The main object of the present invention is to provide a training device for lowering intraocular pressure, which can perform various intraocular pressure lowering trainings for users in a single course of treatment through display headsets, a speaker and a first stimulus medium, so as to achieve the effect of lowering the intraocular pressure of users.

To achieve the above and other objects, a training device for lowering intraocular pressure is provided, wherein the training device for lowering intraocular pressure includes: a bracket unit corresponding to a user's head shape, and comprising a top side part, a back side part and two ears part, wherein the two ears part respectively correspond to the user's ears; two ends of the top side part are respectively connected with the two ears part and straddle the user's head upwards; and two ends of the back side part are respectively connected with the two ears part and straddle the user's back head backwards; a virtual reality unit comprising a display headset connected to the bracket unit and capable of switching between a display mode and an optical frequency stimulation mode, wherein when the display headset is in the display mode, the display headset displays a virtual image, and when the display headset is in the optical frequency stimulation mode, the display headset display a flashing picture; a binaural audio stimulation unit comprising paired speakers, wherein the paired speakers are respectively connected to the two ears part and used for playing binaural beats music, characterized by the audio frequency difference of the binaural beats music is gradually decreased from 18 Hz to 4 Hz, to lower intraocular pressure; a transcranial stimulation unit comprising two first stimulus medium, wherein the two first stimulus medium are respectively connected to the top side part to correspond to F3 position and F4 position among the electrode positions of the International 10-20 EEG system; a control unit electrically connected with the display headset, the speaker and the first stimulus medium, wherein the control unit stores programmed data information of the binaural beats music and the virtual image.

In some embodiments, the first stimulus medium is a first magnetic stimulation medium and is used for outputting electromagnetic pulses to the head of the user for transcranial magnetic stimulation;

The first magnetic stimulation medium comprises an extension frame, a mounting seat and a magnetic coil, wherein the extension frame is connected with the bracket unit, the mounting seat has a first end and a second end which are opposite, the first end is connected with the extension frame, and the magnetic coil is connected with the mounting seat and electrically connected with the control unit.

Preferably, the mounting seat comprises a plurality of support layers stacked in sequence along the direction from the first end to the second end, and each support layer is provided with the magnetic coil; wherein, among any two adjacent support layers, the outer contour of one support layer close to the first end is larger than that of one support layer far from the first end, and the one support layer far from the first end is arranged in the middle of one support layer close to the first end.

Preferably, the end face of one end of the support layer far from the first end is a first magnetic surface, and a first area is surrounded by the magnetic coil around the center of the first magnetic surface on the first magnetic surface; among any two adjacent support layers, one support layer far from the first end is arranged in the first area of one support layer close to the first end.

Preferably, the control unit can control the magnetic coil to emit a plurality of current waves within a predetermined time T, each current wave has a rated current value, and the rated current values of the current waves within the predetermined time are gradually increasing from small to large, and the rated current values are between 1 A-10 A.

In some embodiments, the first stimulus medium is a first electrical stimulation medium, which comprises a connecting seat and a conductive post, the first electrical stimulation medium has a third end and a fourth end which are opposite, the third end is connected to the bracket unit, and the conductive column is connected to the fourth end and electrically connected to the control unit; The number of the conductive columns is multiple, and the conductive columns are distributed on the end face of the fourth end in parallel; The conductive column comprises a connecting part and a silicon electrode part, wherein the connecting part is provided with a fifth end and a sixth end which are opposite, the fifth end is connected to the fourth end, and the silicon electrode part is connected to the sixth end; The number of the silicon electrode parts is multiple, and the silicon electrode parts are distributed on the end face of the sixth end in parallel.

In some embodiments, the transcranial stimulation unit further comprises two second stimulus medium, which are electrically connected with the control unit, and are connected to the top side part and used for outputting current to F7-T3 position and F8-T4 position the among the electrode positions of the International 10-20 EEG system of the user for transcranial electrical stimulation or outputting electromagnetic pulses for transcranial magnetic stimulation.

In some embodiments, it also comprises an eye mask unit, which comprises an eye mask body, two eyes surrounding heating parts and eyes surrounding acupoint stimulation parts, wherein the eye mask body is detachably connected to the bracket unit and straddles the eyes of the user forward; the eye mask body is provided with eye holes for exposing the eyes of the user; the eyes surrounding heating parts are electrically connected with the control unit; and the eyes surrounding heating parts are connected to the eye mask body and used for thermally stimulating the periphery of the eyes of the user through a first preset temperature; The eyes surrounding acupoint stimulation parts are electrically connected with the control unit, and eyes surrounding acupoint stimulation parts are connected with the eye mask body to respectively correspond to two Sibai ST2 acupoints, two Cuanzhu BL2 acupoints and two Qiuhou Ex-HN7 acupoints of the user, and the eyes surrounding acupoint stimulation parts are used for thermally stimulating the two Sibai ST2 acupoints, two Cuanzhu BL2 acupoints and two Qiuhou Ex-HN7 acupoints of the user through a second preset temperature.

In some embodiments, it also comprises an head laser acupoint stimulation unit, which comprises two head laser acupoint stimulation media, two foot sleeves and four foot laser acupoint stimulation media, wherein the head laser acupoint stimulation media and the foot laser acupoint stimulation media are both electrically connected with the control unit, and the two head laser acupoint stimulation media are connected to the back side part to respectively correspond to the two Fengchi GB20 acupoints of the user. And the two head laser acupoint stimulation medium are respectively used for physically stimulating the laser output from the two Fengchi GB20 acupoints of the user, the two foot sleeves are respectively worn by the user's feet, each foot sleeve is provided with two laser stimulation medium of foot acupoint, the four laser stimulation medium of foot acupoint respectively correspond to the user's two Pucan BL61 acupoints and two Shenmai BL62 acupoints, and the four laser stimulation medium of foot acupoint are respectively used for physically stimulating the laser output from the user's two Pucan BL61 acupoints and two Shenmai BL62 acupoints.

Preferably, the audio frequency difference of the binaural beats music is 4 Hz-18 Hz, the laser wavelengths of the head laser acupoints stimulation medium and the foot laser acupoints stimulation medium are 650 nm-808 nm, and the flashing picture is green light with a flashing rate of 5 Hz-50 Hz.

In some embodiments, the system further comprises a exercise bike unit, including a bike frame, a flywheel, a pedal group power module, and an energy conversion module. The bike frame is used for the user to ride, the flywheel is rotatably connected to the bike frame, the pedal group is connected to the flywheel for transmission and for the user to step on the flywheel to rotate it around the central axis of the exercise bike. The energy conversion module is respectively connected to the exercise bike and the power module. The energy conversion module is used to convert the rotational potential energy of the exercise bike into electrical energy and store the electrical energy in the power module. The power module is electrically connected to the control unit.

Compared with the prior art, the training device for lowering intraocular pressure provided by the embodiment of the invention has the beneficial effects that: The display headset can be used for image training and illumination stimulation of users' eyes; the speaker can be used for playing binaural beats music with audio frequency difference to users' ears for music stimulation; the transcranial stimulation unit can output current to F3 position and F4 position of users' heads for transcranial electrical stimulation or output electromagnetic pulse for transcranial magnetic stimulation; therefore, the training device for lowering intraocular pressure provided by the invention can perform various intraocular pressure lowering trainings for users in a single course of treatment through the display headset, the speaker and the first stimulus medium, so as to achieve the synergistical effect of reducing the intraocular pressure of users.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a schematic structural diagram of a training device for lowering intraocular pressure provided by the first embodiment of the present invention;

FIG. 2 is a schematic diagram of the control connection structure of a training device for lowering intraocular pressure provided by the first embodiment of the present invention;

FIG. 3 is a partial structural schematic diagram of a training device for lowering intraocular pressure provided by the first embodiment of the present invention;

FIG. 4 is a side view of a training device for lowering intraocular pressure provided by the first embodiment of the present invention;

FIG. 5 is a front view of a training device for lowering intraocular pressure provided by the first embodiment of the present invention;

FIG. 6 is a schematic structural diagram of a first stimulus medium provided in embodiment 1 of the present invention;

FIG. 7 is a waveform diagram of the current applied by the magnetic coil of the control unit provided in the first embodiment of the present invention.

FIGS. 8a-8d are display diagrams of four kinds of guiding eye training movements of display headset provided by the first embodiment of the present invention, in which FIG. 8a illustrates the state that the guiding point moves upward, FIG. 8b illustrates the state that the guiding point moves upward to the right, FIG. 8c illustrates the state that the guiding point moves rightward, and FIG. 8d illustrates the state that the guiding point moves downward to the right;

FIGS. 9a-9d are display diagrams of four other eye training exercises provided by the first embodiment of the present invention. FIG. 9a illustrates the state that the guiding point moves downward, FIG. 9b illustrates the state that the guiding point moves downward to the left, FIG. 9c illustrates the state that the guiding point moves leftward, and FIG. 9d illustrates the state that the guiding point moves upward to the left.

FIGS. 10a-10c are display diagrams of three other eye training exercises provided by the first embodiment of the present invention, in which, FIG. 10a illustrates the state that the pointer rotates counterclockwise, FIG. 10b illustrates the state that the pointer rotates clockwise, and FIG. 10c illustrates the state that winking is indicated;

FIG. 11 is a diagram showing the effect relationship between various intraocular pressure training and the percentage of intraocular pressure reduction after training by using an intraocular pressure reduction training device provided by the first embodiment of the present invention; and

FIG. 12 is a schematic structural diagram of the first stimulus medium provided by the second embodiment of the present invention.

### DETAILED DESCRIPTION OF THE PREFERRED EMBODIMENTS

In the following, the specific embodiments of the present invention will be described in further detail with reference to the attached drawings and examples. The following examples are used to illustrate the invention, but are not used to limit the scope of the invention.

In the description of the present invention, it should be understood that the terms “center”, “transverse”, “length”, “width”, “thickness”, “above”, “below”, “front”, “back”, “left”, “right”, “vertical”, “horizontal”, “top”, “bottom”, “inside”, “outside” and other directional or positional relationships indicated are based on the directional or positional relationships shown in the accompanying drawings, only for the convenience of describing the present invention and simplifying the description, and do not indicate or imply that the device or element referred to must have a specific orientation, be constructed and operated in a specific orientation, and therefore cannot be understood as a limitation of the present invention.

The terms “first” and “second” are only used for descriptive purposes, and cannot be understood as indicating or implying relative importance or implicitly indicating the number of indicated technical features. Therefore, the features defined as “first” and “second” can include one or more features explicitly or implicitly.

In the present invention, unless otherwise specified and limited, the terms “installation”, “connect”, “connection” and “fixation” should be broadly understood, for example, they can be fixed connection, detachable connection or integrated. It can be a mechanical connection or an electrical connection, can be directly connected, can also be indirectly connected through an intermediary, can be the internal communication of two elements or the interaction between two elements. For those skilled in the art, the specific meanings of the above terms in the present invention can be understood according to specific situations.

In the present invention, unless otherwise specified and limited, the first feature “above” or “below” the second feature may include direct contact between the first and second features, or it may include that the first and second features are not in direct contact but through another feature contact between them. Moreover, the words “above”, “above” and “above” of the first feature include that the first feature is directly above and obliquely above the second feature, or only indicate that the horizontal height of the first feature is higher than the second feature. The first feature being “below”, “below”, and “below” include the first feature being directly below and diagonally below the second feature, or simply indicating that the first feature has a lower horizontal height than the second feature.

### Embodiment 1

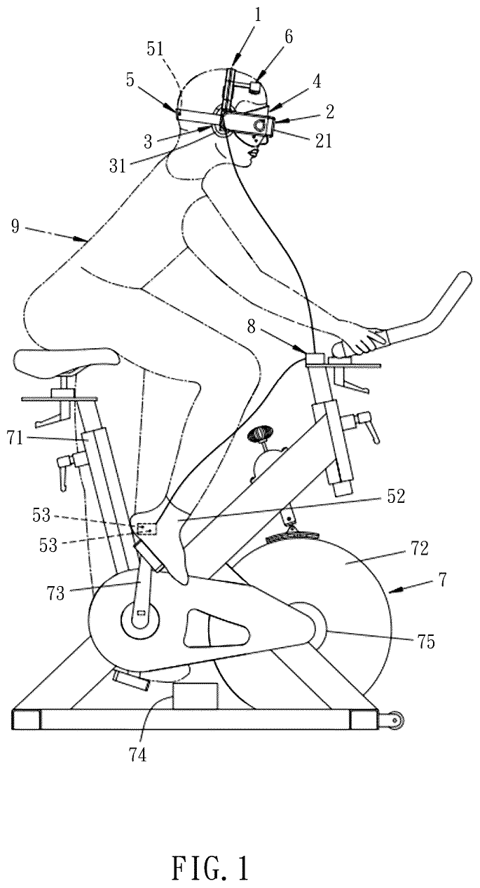

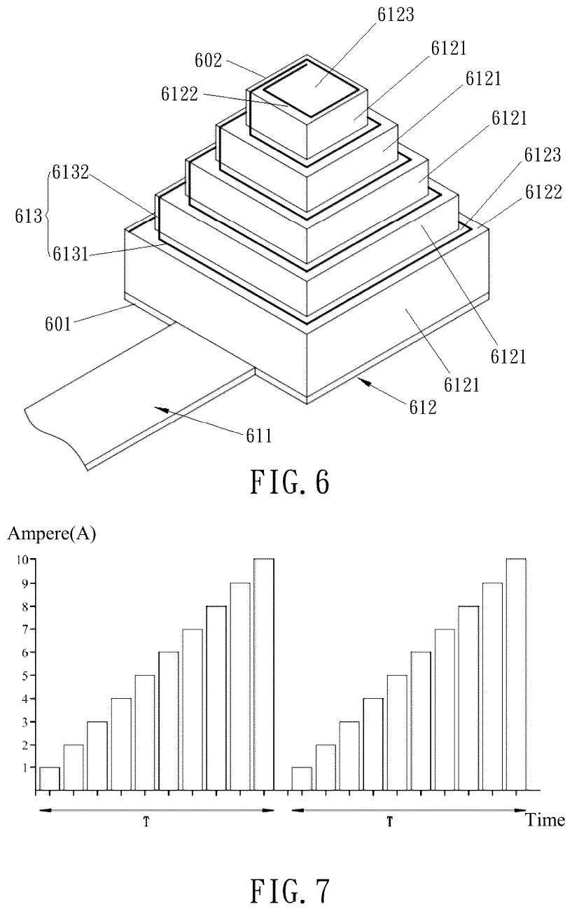

As shown in FIGS. 1-7, a training device for lowering intraocular pressure preferred by an embodiment of the present invention includes a bracket unit 1, a virtual reality unit 2, a binaural audio stimulation unit 3, an eye mask unit 4, an head laser acupoint stimulation unit 5, a transcranial stimulation unit 6, an exercise bike unit 7 and a control unit 8.

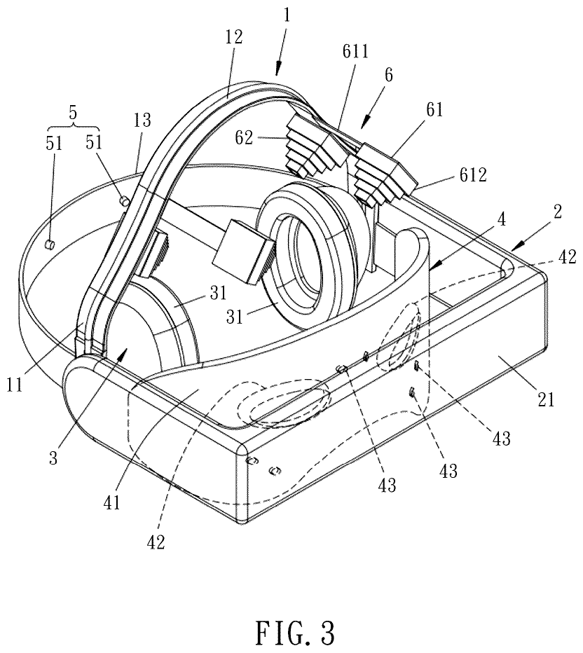

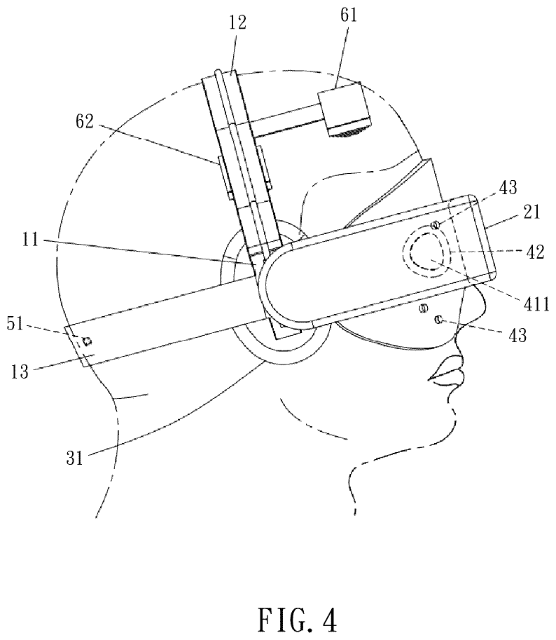

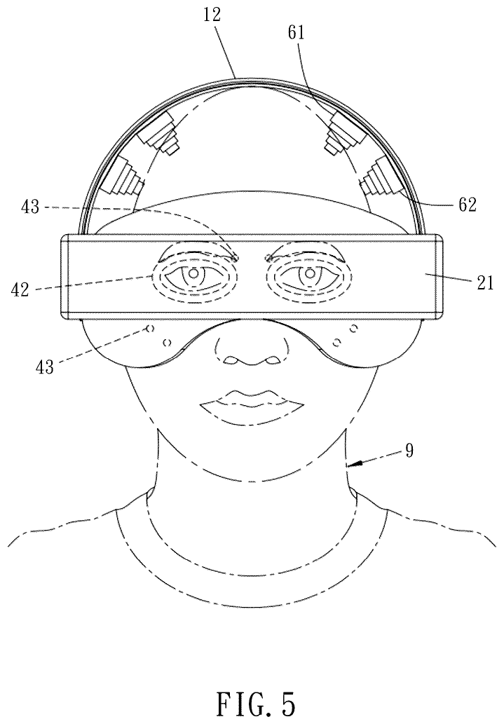

Referring to FIG. 1, FIG. 3, FIG. 4 and FIG. 5, the bracket unit 1 corresponds to a user's head shape, and the bracket unit 1 includes a top side part 12, a back side portion 13 and two ears part 11, wherein the two ears part 11 correspond to the user's ears respectively; the two ends of the top side part 12 are respectively connected with the two ears part 11 and straddle the user's head upwards; and the two ends of the back side portion 13 are respectively connected with the two ears part 11 and straddle the user's back head backwards.

Referring to FIGS. 1-5, the virtual reality unit 2 includes display headset 21, which are connected to the bracket unit 1 and can switch between the display mode and the light frequency stimulation mode. When the display headset 21 are in the display mode, the display headset 21 display virtual images for the eyes of the user to watch, and when the display headset 21 are in the light frequency stimulation mode, the display headset 21 stimulate the eyes of the user with flashing pictures. The display headset 21 can play virtual images in the display mode to guide users' eyeballs to track and move, so as to achieve the auxiliary effect of vision training for lowering intraocular pressure, and can play flashing pictures in the light frequency stimulation mode to stimulate users' eyes, so as to achieve the auxiliary effect of vision training for lowering intraocular pressure.

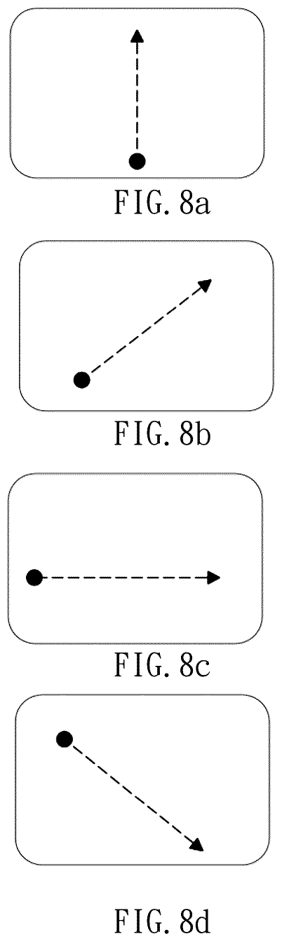

In this embodiment, the display headset 21 can display eleven virtual images of eye movement training as shown in FIGS. 8a-10c to guide the user to do eye movements. Of course, the display headset 21 can also display virtual images in any form, such as light spots and patterns, for the user to carry out eye movement training, but it is not limited thereto.

In this embodiment, the flashing picture is green light with a flashing rate of 5 Hz-50 Hz. That is, when the display headset 21 are in the light frequency stimulation mode, the display headset 21 display green light with a flicker rate of 5 Hz-50 Hz.

The binaural audio stimulation unit 3 includes paired speakers 31, which are respectively connected to the two ears part 11. Binaural beats music with a specific audio frequency difference can be played through the speaker 31, so as to induce the user's brain waves to induce frequency following response and enter a specific brain wave form to achieve lower intraocular pressure via binaural beats music.

Preferably, the speaker 31 is used to play a Binaural beats with Frequency Following Response to the ears of the user 9, and the binaural beats music has an audio frequency difference between 4 Hz-18 Hz, which gradually decreases the audio frequency difference from 18 Hz to 4 Hz.

Referring to FIG. 1, FIG. 3, FIG. 4 and FIG. 5, the eye mask unit 4 includes an eye mask body 41, two eyes surrounding heating parts 42 and eyes surrounding acupoint stimulation parts 43, wherein the eye mask body 41 is detachably connected to the bracket unit 1 and straddles the user's eyes forward; the two eyes surrounding heating parts 42 are electrically connected with the control unit 8; the eye mask body 41 is provided with eye holes 411 for the eyes of the user 9 to be exposed; and the two eyes surrounding heating parts 42 are connected to the eye mask body 41 and used for heating the eyes of the user through a first preset temperature; The eyes surrounding acupoint stimulation parts 43 are electrically connected to the control unit 8, and the eyes surrounding acupoint stimulation parts 43 are connected to the eye mask body 41 to correspond to the user's two Sibai ST2 acupoints, two Cuanzhu BL2 acupoints and two Qiuhou Ex-HN7 acupoints respectively, and the eyes surrounding acupoint stimulation parts 43 are used to thermally stimulate the user's two Sibai ST2 acupoints, two Cuanzhu BL2 acupoints and two Qiuhou Ex-HN7 acupoints respectively at a second preset temperature. Through the eye mask unit 4, when the user 9 performs eye training or illumination stimulation through the display headset 21, at the same time, the two eyes surrounding heating element 42 and the eyes surrounding acupoint stimulation parts 43 can respectively perform thermal stimulation on the periphery of the user's eyes and specific eye acupoints, so as to stimulate the nerves around the eyes, relax the eye muscles, and promote the blood circulation around the eyes to achieve the auxiliary effect of vision training for lowering intraocular pressure.

In this embodiment, both ends of the eye mask body 41 are detachably connected to the two ears part 11. The width length of two eyes surrounding heating parts 42 can be 1-3 cm.

In this embodiment, the first preset temperature is between 40 °C and 45 °C.

In this embodiment, the second preset temperature is 50 °C.

Preferably, the two eyes surrounding heating parts 42 is made of graphene, and the eyes surrounding acupoint stimulation parts 43 is made of graphene or low-level laser.

Referring to FIGS. 1-5, the head laser acupoint stimulation unit 5 includes two head laser acupoint stimulation medium 51, two foot covers 52, and four laser stimulation medium of foot acupoint 53. The head laser acupoint stimulation medium 51 and the laser stimulation medium of foot acupoint 53 are both electrically connected to the control unit 8. The two head laser acupoint stimulation medium 51 are connected to the back side part 13 to correspond to the user's two Fengchi acupoints respectively, and the two head laser acupoint stimulation medium 51 are used to output laser for physical stimulation to the user's two Fengchi acupoints respectively. The two foot covers 52 are respectively worn by the user's feet, and each foot cover 52 is equipped with two laser stimulation medium of foot acupoint 53. The four laser stimulation medium of foot acupoint 53 are used to apply laser to the user's two Pucan BL61 acupoints respectively. The acupoint and two Shenmai BL62 acupoints output laser for physical stimulation. The head laser acupoints stimulation medium 51 and the foot laser acupoints stimulation medium 53 can respectively perform laser stimulation on specific head acupoints and specific foot acupoints, so as to stimulate vision-related acupoints and further achieve the auxiliary effect of training device for lowering intraocular pressure.

In this embodiment, the laser wavelengths of the head laser acupoints stimulation medium 51 and the foot laser acupoints stimulation medium 53 are 808 nm.

Referring to FIGS. 3-6, the transcranial stimulation unit 6 includes two first stimulus mediums 61 and two second stimulus mediums 62. The two first stimulus mediums 61 are respectively connected to the top side part 12 to correspond to the F3 position and F4 position of the international 10-20 electroencephalogram electrode, and the first stimulus mediums 61 are used for outputting current to the F3 position and F4 position of the user's head for transcranial electrical stimulation or electromagnetic pulse for transcranial magnetic stimulation. The second stimulus medium 62 is connected to the top side part 12 and is used to output current to the F7-T3 position and F8-T4 position of the user for transcranial electrical stimulation or output electromagnetic pulse for transcranial magnetic stimulation. The transcranial stimulation unit can output current to F3 position, F4 position and F7-T3 position, F8-T4 position of the user's head for transcranial electrical stimulation or output electromagnetic pulse for transcranial magnetic stimulation.

In this embodiment, the first stimulus medium 61 is the first magnetic stimulation medium and is used to output electromagnetic pulses to the head of the user for transcranial magnetic stimulation. The first magnetic stimulation medium includes an extension frame 611, a mounting seat 612 and a magnetic coil 613. The extension frame 611 is connected to the bracket unit 1, and the mounting seat 612 has a first end 601 and a second end 602 opposite to each other. The first end 601 is connected to the extension frame 611, and the magnetic coil 613 is arranged on the mounting seat 612. The magnetic coil 613 is electrically connected with the control unit 8 and can generate magnetic force when power is applied.

In this embodiment, the extension frame 611 is connected to the top side part 12.

The mounting seat 612 includes a plurality of support layers 6121 stacked in sequence along the direction from the first end 601 to the second end 602, and each support layer 6121 is provided with a magnetic coil 613. Among any two adjacent support layers 6121, the outer contour of one support layer 6121 near the first end 601 is larger than that of one support layer 6121 far from the first end 601, and the one support layer 6121 far from the first end 601 is arranged in the middle of one support layer 6121 near the first end 601. In this way, the mounting seat 612 can be made into a laminated tower structure that gradually narrows along the first end 601 to the second end 602, so that the support layer 6121 farthest from the first end 601 (that is, the support layer 6121 located at the end of the second end 602) can be aligned with the F3 position or F4 position of the user, so that the magnetic force of the magnetic coil 613 can accurately stimulate the F3 position or F4 position of the user through the second end 602, so as to effectively reduce the intraocular pressure of the user.

In this embodiment, the number of support layers 6121 is five, and the five support layers 6121 are the first support layer, the second support layer, the third support layer, the fourth support layer and the fifth support layer which are sequentially distributed along the direction from the first end 601 to the second end 602, so the outer contour of the first support layer is larger than that of the second support layer, and the second support layer is arranged in the middle of the first support layer. The outer contour of the second supporting layer is larger than that of the third supporting layer, and the third supporting layer is arranged in the middle of the second supporting layer. The outer contour of the third supporting layer is larger than that of the fourth supporting layer, and the fourth supporting layer is arranged in the middle of the third supporting layer. The outer contour of the fourth support layer is larger than that of the fifth support layer, and the fifth support layer is arranged in the middle of the fourth support layer.

In this embodiment, the support layer 6121 has a square structure, and the length, width and height of the support layer 6121 with the largest outer contour are 3 cm, 3 cm and 1 cm respectively. The supporting layer 6121 with the smallest outer contour is 1 cm in length, 1 cm in width and 1 cm in height. It can be adapted to the F3 position and F4 position of the head of the user 9, and effectively magnetically stimulate the F3 position and F4 position of the head of the user 9, so as to effectively reduce the intraocular pressure of the user 9.

The end face of the support layer 6121 far from the first end 601 is a first magnetic surface 6122, and the magnetic coil 613 surrounds the center of the first magnetic surface 6122 on the first magnetic surface 6122 with a first area 6123. Among any two adjacent support layers 6121, one support layer 6121 far from the first end 601 is arranged in the first area 6123 of one support layer 6121 near the first end 601. In this way, the first magnetic coil 613 can be arranged in a spiral shape around a plurality of support layers 6121 to concentrate electromagnetic pulses, and the first magnetic surface 6122 of one support layer 6121 (i.e., the fifth support layer) located on the second end 602 can be adapted to the P1 position or P2 position of the user, so that the second end 602 can accurately stimulate the concentrated electromagnetic pulses to the F3 position or F4 position of the user, so as to reduce the intraocular pressure of the user more effectively.

Specifically, in any two adjacent support layers 6121, the magnetic coil 613 is arranged around the outer contour of the support layer 6121 away from the first end 601 on the first magnetic surface 6122 of the support layer 6121 near the first end 601, and extends from the first magnetic surface 6122 of the support layer 6121 near the first end 601 along the outer wall of the support layer 6121 away from the first end 601 to the first magnetic surface 6122 of the support layer 6121 away from the first end 601.

More specifically, the magnetic coil 613 includes multiple surrounding segments 6131 and multiple straight line segments 6132. The multiple surrounding segments 6131 and straight line segments 6132 are alternately connected, and the surrounding segments 6131 are set on the first magnetic surface 6122 and enclose the first area 6123. The straight line segments 6132 are set on the outer wall of the support layer 6121, and the straight line segments 6132 are the shortest interconnection structure connecting the two first magnetic surfaces 6122. In this way, it can be adapted to the stacked tower like structure of the installation base 612, and the magnetic coil 613 can be designed in a spiral like structure that gradually narrows along the direction from the first end 601 to the second end 602 to concentrate electromagnetic pulses along the direction from the first end 601 to the second end 602. It can also have the shortest interconnection path between adjacent surrounding segments 6131 to reduce the energy loss of the magnetic coil 613 and more effectively and stably convert electricity into magnetic force, thereby enabling more effective magnetic stimulation of the F3 or F4 position of the user and more effectively reducing the user's intraocular pressure.

In this embodiment, the second stimulus medium 62 is a second magnetic stimulation medium to F7-T3 position and F8-T4 position, and the structure of the second magnetic stimulation medium is similar to that of the first magnetic stimulation medium, so further explanation is not given.

Refer to Table 1 below, the intraocular pressure values obtained by tonometer detection for 10 myopia users, in which each user uses one unit of the intraocular pressure training device provided by this embodiment once for 30 minutes by listening to binaural beats music and stimulating the F7-T3 position and F8-T4 position of the user. The intraocular pressure values before training in Table 1 are those measured by the user before using the intraocular pressure training device provided by this embodiment, and the intraocular pressure values after training in Table 1 are those after using the intraocular pressure training device provided by this embodiment. As shown in the following table 1:

TABLE 1

| | | Intraocular pressure | Intraocular pressure |
|---|---|---|---|
| User | Use time | before training (mmHg) | after training (mmHg) |
| 1 | 30 min | 28 | 24 |
| 2 | 30 min | 27 | 22 |
| 3 | 30 min | 26 | 17 |
| 4 | 30 min | 24 | 18 |
| 5 | 30 min | 23 | 19 |
| 6 | 30 min | 23 | 18 |
| 7 | 30 min | 23 | 17 |
| 8 | 30 min | 22 | 18 |
| 9 | 30 min | 19 | 13 |
| 10 | 30 min | 17 | 15 |

As can be seen from Table 1, the average intraocular pressure of 10 users with myopia and high intraocular pressure before training is 23 mmHg, and the average intraocular pressure of 10 users after training decreases by 5 mmHg after listening to binaural beats music with frequency difference from 18 Hz to 4 Hz and stimulating the F7-T3 position, F8-T4 position, two units of the training device for lowering intraocular pressure provided by the embodiment of the present invention.

Refer to Table 2 below, the intraocular pressure values obtained by tonometer detection for 10 myopia users, in which each user uses the intraocular pressure training device provided by this embodiment once for 30 minutes by listening to binaural beats music and stimulating the F3 position, F4 position and F7-T3 position, F8-T4 position of the user's head. The intraocular pressure values before training in Table 2 are those measured by users before using the intraocular pressure training device provided by this embodiment, and the intraocular pressure values after training in Table 2 are those measured by users after using the intraocular pressure training device provided by this embodiment, as shown in the following table 2:

TABLE 2

| | | Intraocular pressure | Intraocular pressure |
|---|---|---|---|
| User | Use time | before training (mmHg) | after training (mmHg) |
| 1 | 30 min | 28 | 21 |
| 2 | 30 min | 27 | 20 |
| 3 | 30 min | 26 | 15 |
| 4 | 30 min | 24 | 16 |
| 5 | 30 min | 23 | 17 |
| 6 | 30 min | 23 | 15 |
| 7 | 30 min | 23 | 15 |
| 8 | 30 min | 22 | 17 |
| 9 | 30 min | 19 | 10 |
| 10 | 30 min | 17 | 15 |

As can be seen from Table 2, after listening to binaural beats music and stimulating F3 position, F4 position and F7-T3 position, F8-T4 position by using the training device for lowering intraocular pressure provided by the embodiment of the present invention, the intraocular pressure of 10 myopia users and have high original intraocular pressure values between 17 mmHg and 28 mmHg decreased by 7 mmHg on average.

Refer to Table 3 below, the intraocular pressure values obtained by tonometer detection for 10 presbyopia users, in which each user uses the intraocular pressure training device provided by this embodiment once for 30 minutes by listening to binaural beats music with frequency difference from 18 Hz to 4 Hz and stimulating the F3 position, F4 position and F7-T3 position, F8-T4 position of the user's head. The intraocular pressure values before training in Table 3 are those measured by users before using the intraocular pressure training device provided by this embodiment, and the intraocular pressure values after training in Table 3 are those measured by users after using the intraocular pressure training device provided by this embodiment, as shown in the following table 3:

TABLE 3

| | | Intraocular pressure | |
|---|---|---|---|
| | | before training | Intraocular pressure after |
| User | Use time | (mmHg) | training (mmHg) |
| 1 | 30 min | 25 | 18 |
| 2 | 30 min | 25 | 16 |
| 3 | 30 min | 24 | 20 |
| 4 | 30 min | 24 | 19 |
| 5 | 30 min | 24 | 17 |
| 6 | 30 min | 23 | 18 |
| 7 | 30 min | 22 | 17 |
| 8 | 30 min | 21 | 15 |
| 9 | 30 min | 20 | 17 |
| 10 | 30 min | 18 | 17 |

As can be seen from Table 3, after listening to binaural beats music and stimulating F3 position, F4 position and F7-T3 position, F8-T4 position by using the training device for lowering intraocular pressure provided by the embodiment of the present invention, 10 users with presbyopia and high original intraocular pressure values between 18 mmHg-25 mmHg, the intraocular pressure values after training decreased by 5.2 mmHg on average.

Table 1-3 shows that listening to binaural beats music and stimulation of F3 position, F4 position and F7-T3 position, F8-T4 position brain regions can reduce intraocular pressure.

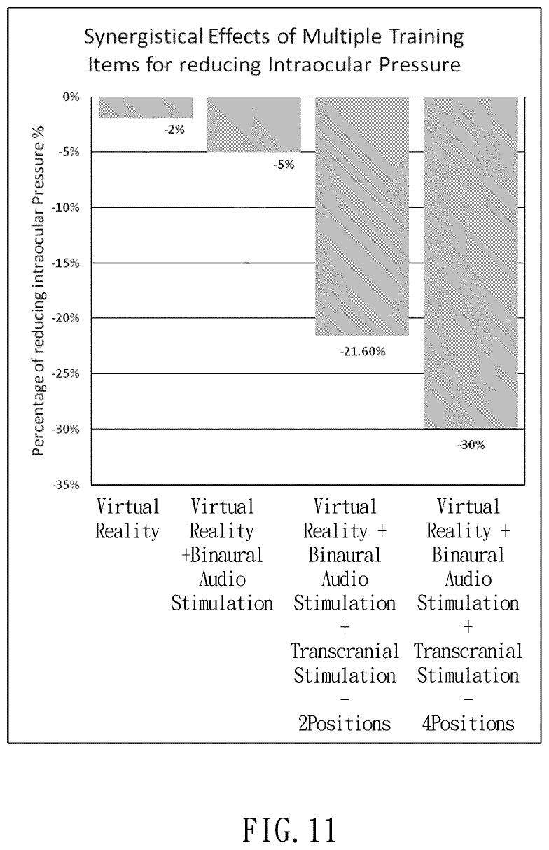

Referring to FIG. 1, FIG. 3, FIG. 4 and FIG. 5, in this application, the virtual reality unit 2, the binaural audio stimulation unit 3, the eye mask unit 4, the head laser acupoint stimulation unit 5 and the transcranial stimulation unit 6 are all connected to the bracket unit 1, so that the bracket unit 1, the virtual reality unit 2, the binaural audio stimulation unit 3, the eye mask unit 4, the head laser acupoint stimulation unit 5 and the transcranial stimulation unit 6 can form a wearable training device which integrates various training items for lowering intraocular pressure. Referring to FIG. 11, the training device for lowering intraocular pressure provided by the first embodiment is applied to the user for intraocular pressure lowering training in a single course of treatment. As can be seen from FIG. 11, the more training items for lowering intraocular pressure, the better lowering effect of the intraocular pressure, that is, multiple training for lowering intraocular pressure can achieve the synergistical effect of reducing the intraocular pressure of the user.

Referring to FIG. 1, the exercise bike unit 7 includes a bike frame 71, a flywheel 72, a pedal group 73, a power module 74, and an energy conversion module 75. The bike frame 71 is used for the user to ride, and the flywheel 72 is rotatably connected to the bike frame 71. The pedal group 73 is connected to the flywheel 72 for the user 9 to step on and rotate the flywheel 72 around the central axis of the exercise bike 7. The energy conversion module 75 is connected to the flywheel 72 and the power module 74 respectively. The energy conversion module 75 is used to convert the rotational potential energy of the flywheel 72 into electrical energy and store it in the power module 74. The power module 74 is electrically connected to the control unit 8.

In this embodiment, the energy conversion module 75 performs energy conversion by means of magnetic power generation, which is the power generation technology of a general generator, so it will not be further described.

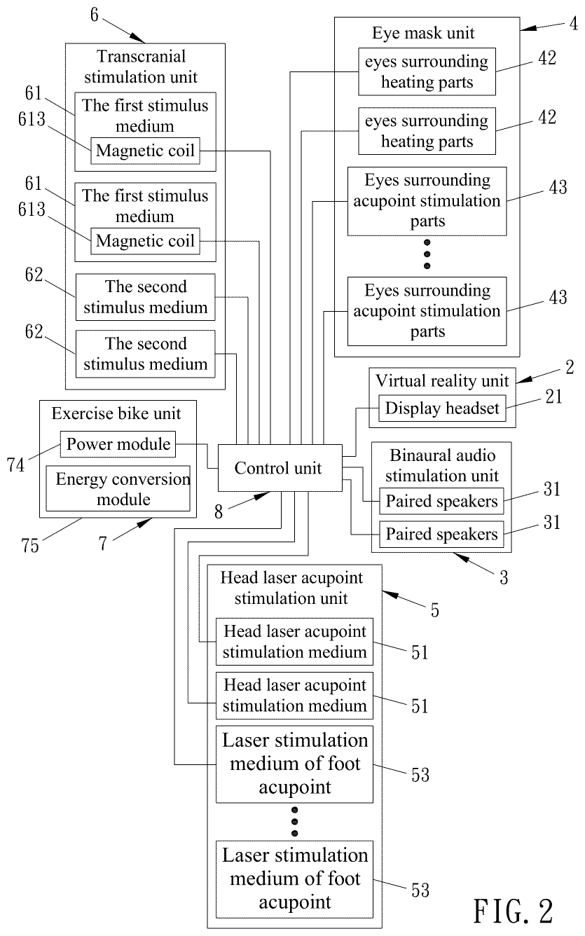

Referring to FIGS. 1 and 2, the control unit 8 is electrically connected with the display headset 21, the speaker 31, the two eyes surrounding heating parts 42, the eyes surrounding acupoint stimulation parts 43, the head laser acupoints stimulation medium 51, the foot laser acupoints stimulation medium 53, the first stimulus medium 61, the second stimulus medium 62 and the power module 74. The control unit 8 stores data information of binaural beats music and virtual images, and can receive power from the power module 74. The control unit 8 can control the display headset 21 to display the virtual image or flashing green light, the control unit 8 controls the two eyes surrounding heating parts 42 and the eyes surrounding acupoint stimulation parts 43 to generate heat, the control unit 8 controls the head laser acupoints stimulation medium 51 and the foot laser acupoints stimulation medium 53 to emit laser, and the control unit 8 controls the first magnetic stimulation medium and the second magnetic stimulation medium to emit magnetic stimulation.

Referring to FIG. 7, in this embodiment, the control unit 8 can control the magnetic coil 613 to emit a plurality of current waves within a predetermined time T, each current wave has a rated current value, and the rated current value of the current waves within the predetermined time is gradually increasing from small to large, and the rated current value is between 1 A-10 A.

In some embodiments, the predetermined time T is 1 second, and the number of current waves is 10 groups, but it can also be adjusted to 20 groups every 1 second as required, without limitation.

In this embodiment, the control unit 8 is arranged on the exercise bike unit 7, but the control unit 8 can also be arranged in the virtual reality unit 2, and is not limited to this.

Stimulation of the corresponding head acupoint by the head laser acupoint stimulation medium 51 and stimulation of the corresponding foot acupoint by the foot laser acupoint stimulation medium 53 can respectively improve the intraocular pressure, which can be proved by the following references, so this specification will not be further explained.
1. Takayama, S., et al., Short-term effects of acupuncture on open-angle glaucoma in retrobulbar circulation: additional therapy to standard medication. Evid Based Complement Alternat Med, 2011. 2011: p. 157090.

2. Liu, W., et al., Impact of acupuncture on 24 h intraocular pressure of glaucoma. Zhongguo zhen jiu=Chinese acupuncture & moxibustion, 2011. 31(6): p. 518-520.
3. Yeh, T. Y., J. C. Lin, and C. F. Liu, Effect of transcutaneous electrical nerve stimulation through acupoints of Pucan (BL 61) and Shenmai (BL62) on intraocular pressure in patients with glaucoma: a randomized controlled trial. J Tradit Chin Med, 2016. 36(1): p. 51-6.
4. Chen, J., et al., Acupuncture Treatment Reverses Retinal Gene Expression Induced by Optic Nerve Injury via RNA Sequencing Analysis. Front Integr Neurosci, 2019. 13: p. 59.

In addition, exercise training and monochromatic green light phototherapy can reduce the incidence of glaucoma, which can be proved by the following references, so this specification will not be further explained.
5. Meier, N. F., et al., Physical Activity, Cardiorespiratory Fitness, and Incident Glaucoma. Med Sci Sports Exerc, 2018. 50(11): p. 2253-2258.
6. Sergienko, N. M. and I. V. Shargorodskaia, Effects of monochromatic light on ocular hydrodynamics. Vestn Oftalmol, 2000. 116(4): p. 17-9.

Referring to FIG. 1-FIG. 10c, the use method of the training device for lowering intraocular pressure provided by the embodiment of the invention is as follows: Before use, the user 9 wears the bracket unit 1 and the foot cover 52, then sits on the bike frame 71 and steps on the pedal group 73 to prepare for the training of pedaling the exercise bike. During training, the user 9 begins to step on the pedal group 73 for 30 minutes. At this time, the control unit 8 controls the first stimulus medium 61 and the second stimulus medium 62 to emit magnetic stimulation to the user's head for 30 minutes, and simultaneously controls the head laser acupoints stimulation medium 51 and the foot laser acupoints stimulation medium 53 to emit laser light stimulation to the corresponding acupoints for 30 minutes, and simultaneously controls the two eyes surrounding heating parts 42 and the eyes surrounding acupoint stimulation parts 43 to heat the eyes and the corresponding acupoints for 30 minutes. In the first 15 minutes, the control unit 8 controls the display headset 21 to switch to the display mode, and controls the speaker 31 to play binaural beats music. In the next 3 minutes, the control unit 8 controls the display headset 21 to switch to the optical frequency stimulation mode, and controls the speaker 31 to stop playing binaural beats music and play health education content instead. In the last 12 minutes, the control unit 8 controls the display of the display headset 21 to switch to the display mode, and controls the speaker 31 to play the binaural beats music, thus completing the whole training.

It should be noted that when the display headset 21 are switched to the light frequency stimulation mode, the health education content played by the speaker 31 includes suggestions on diet, exercise, sleep, environmental arrangement, activity arrangement, etc. The following is a list of relevant contents, but it is not a restrictive condition.

I. Eat to maintain a good intraocular pressure. Drinking a lot of water will quickly increase intraocular pressure. It is recommended to drink a small amount of water many times, and the total amount will remain unchanged. Drinking should be moderate, and wine is recommended. Nicotine is harmful to retina, so smoking must be quitted. Sweet food should be moderate and eat more colored food. Hypertonic foods (honey, sugar, etc.) help to reduce intraocular pressure, so it is recommended to eat them in moderation, but if patients have diabetes, cardiovascular diseases, etc., they must control their consumption. Ingesting some colorful foods, such as wolfberry, blueberry, ginkgo leaf, etc., can protect the optic nerve.

II. Regular exercise improves intraocular pressure. Regular aerobic exercise is good for controlling intraocular pressure. Regular aerobic exercise, such as walking for about 30 minutes, can reduce intraocular pressure.

III. Good sleep improves intraocular pressure. Do not lie on your side as much as possible, and raise your lower limbs appropriately. Lying on the side will increase the intraocular pressure. For glaucoma patients with normal intraocular pressure, attention should be paid to raising the intracranial pressure, such as raising the lower limbs while sleeping, but it is not appropriate to keep upside down.

IV. Environmental layout. Maintain cool and comfortable temperature and humidity in the bedroom (the indoor environment is adjusted to 23 degrees Celsius and the relative humidity is maintained at 40% to 70%). Keep the bedroom lights dim. Keep the environment quiet. The phone turns to mute. Choose comfortable sleeping bed.

V. Activity arrangement. Get up in the sun (receive sunlight stimulation). Try to relax after work. Listen to relaxing music or binaural beats music before going to bed. Watch the natural dim picture (or the screen shows the night starlight) before bed. Do not exercise excessively, eat midnight snack or eat irritating food for 3 hours before bed.

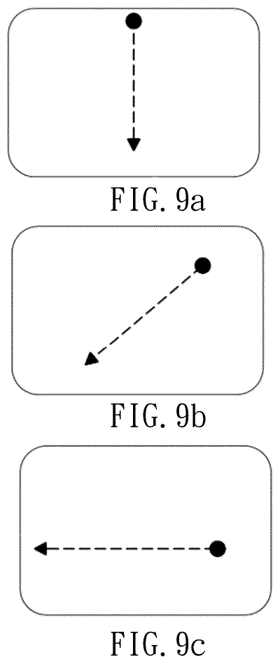

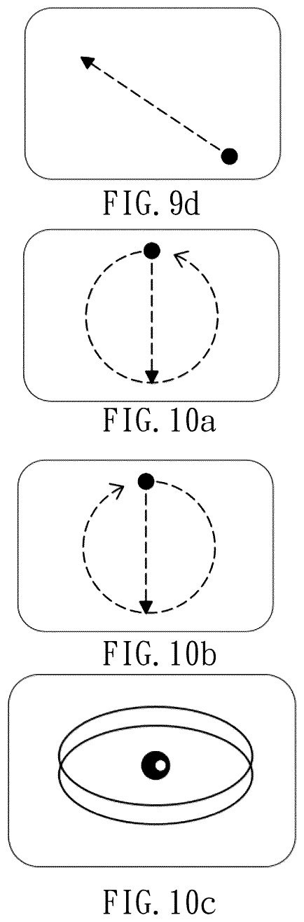

When the display headset 21 are switched to the image mode, the displayed image shows the eye movement training, as shown in FIGS. 8a-8d, 9a-9d and 10a-10c, in this embodiment, it is presented in the way that the guiding point moves in the picture, so that the user can change the eyeball position with the guiding point. There are eleven types of eye movements, including upward movement (see FIG. 8a), upward right movement (see FIG. 8b), downward right movement (see FIG. 8c), downward right movement (see FIG. 8d), downward left movement (see FIG. 9a), downward left movement (see FIG. 9b), upward left movement (see FIG. 9c), counterclockwise rotation (see FIG. 10a), clockwise rotation (see FIG. 10b), and blinking (see FIG. 10c).

To sum up, the training device for lowering intraocular pressure provided by the first embodiment of the present invention is provided with a virtual reality unit 2, a speaker 31 for playing binaural beats music, an head laser acupoint stimulation unit 5 for providing acupoint stimulation, a transcranial stimulation unit 6 for providing magnetic stimulation, an eye mask unit 4 for providing thermal stimulation, and a exercise unit 7 for training, so that users can carry out various intraocular pressure lowering trainings for users in a single course of treatment, and the effect of lowering the intraocular pressure of users can be achieved.

### Embodiment 2

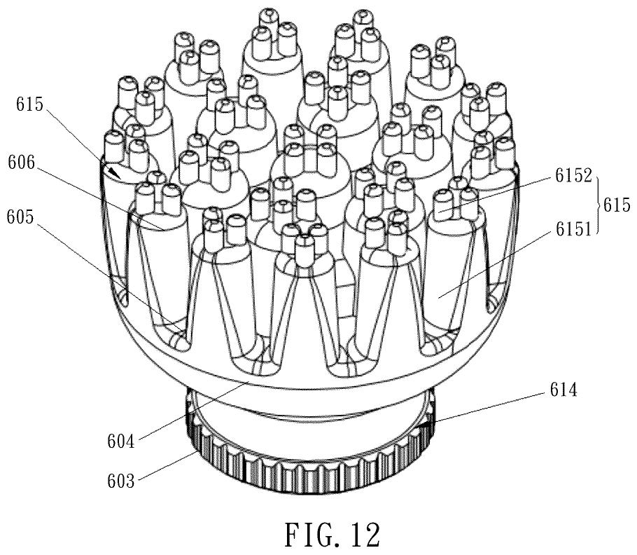

Different from the first embodiment, referring to FIG. 12, the first stimulus medium 61 provided by the second embodiment is the first electrical stimulation medium.

The first stimulus medium 61 is the first electrical stimulation medium and is used to output current to F3 position and F4 position of the user's head for transcranial electrical stimulation. The first electrical stimulation medium is in the form of outputting direct or alternating current, and the current of the direct or alternating current is less than 2 mA.

In this embodiment, the first electrical stimulation medium includes a connecting set 614 and a conductive column 615. The connecting set 614 has a third end 603 and a fourth end 604 which are opposite to each other. The third end 603 is connected to the bracket unit 1, and the conductive column 615 is connected to the fourth end 604 and electrically connected to the control unit 8.

The number of conductive posts 615 is multiple, and the plurality of conductive posts 615 are distributed in parallel on the end face of the fourth end 604. A plurality of conductive posts 615 are connected to the end face of the connecting seat 614, so that a single conductive column 615 can pass through the hair with a smaller volume, so that the single conductive column 615 has a smaller and more contact area with the user 9, and the smaller and more contact area can reduce the current density passing through the skin, and the current is extremely small without stimulation risk or discomfort. At the same time, the plurality of conductive posts 615 can form a plurality of contact positions with the user 9 and form a larger coverage area, thereby ensuring that the first electrical stimulation medium can cover the F3 position or F4 position of the user 9 of the user.

In this embodiment, the end face of the fourth end 604 has a circular structure, and the center of the end face of the fourth end 604 is provided with a conductive column 615. The end face of the fourth end 604 has a plurality of arrangement areas distributed at intervals along its radial direction, and each arrangement area has a plurality of conductive columns 615 distributed around the center of the end face of the fourth end 604. In this way, a plurality of conductive posts 615 can be densely arranged on the end face of the circular fourth end 604 to form a circular coverage area, so that the first electrical stimulation medium can completely cover the area to be stimulated by the user 9 and make part of the conductive posts 615 accurately aim at the F3 position or F4 position of the user 9, so that the current of the conductive posts 615 can accurately stimulate the F3 position or F4 position of the user to effectively reduce the intraocular pressure of the user.

Specifically, in this embodiment, the number of arrangement regions is two.

In other embodiments, the number of arrangement regions can also be any number such as one, three or four, which is not limited here.

The conductive column 615 comprises a connecting part 6151 and a silicon electrode part 6152, wherein the connecting part 6151 has a fifth end 605 and a sixth end 606 which are opposite, the fifth end 605 is connected to the fourth end 604, and the silicon electrode part 6152 is connected to the sixth end 606. The number of silicon electrode portions 6152 is plural, and the plurality of silicon electrode portions 6152 are distributed in parallel on the end face of the sixth end 606. The adoption of a plurality of silicon electrode parts 6152 enables a single silicon electrode part 6152 to have a smaller volume, so that the single silicon electrode part 6152 has a smaller contact area with the user 9, and the smaller and more contact area can reduce the current density through the skin, and the current is extremely small without stimulation risk or discomfort. At the same time, because the material of the silicone electrode part 6152 is soft, the silicone electrode part 6152 can be in soft contact with the user 9, which is helpful to further reduce the discomfort.

The silicon electrode part 6152 is made of silicon electrode material.

The material of the connecting part 6151 and the material of the silicon electrode part 6152 may be the same or different.

The above is only the preferred embodiment of the present invention, and it should be pointed out that a person skilled in the art can make several improvements and substitutions without departing from the technical principles of the present invention, and these improvements and substitutions should also be regarded as the protection scope of the present invention.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A training device for lowering intraocular pressure including:
a bracket unit corresponding to a user's head shape, and comprising a top side part, a back side part and two ears part, wherein the two ears part respectively correspond to the user's ears; two ends of the top side part are respectively connected with the two ears part and straddle the user's head upwards; and two ends of the back side part are respectively connected with the two ears part and straddle the user's back head backwards;
a virtual reality unit comprising a display headset connected to the bracket unit and capable of switching between a display mode and an optical frequency stimulation mode, wherein when the display headset is in the display mode, the display headset displays a virtual image, and when the display headset is in the optical frequency stimulation mode, the display headset displays a flashing picture;
a binaural audio stimulation unit comprising paired speakers, wherein the paired speakers are respectively connected to the two ears part and used for playing binaural beats music, characterized by the audio frequency difference of the binaural beats music is gradually decreased from 18 Hz to 4 Hz, to lower intraocular pressure;
a transcranial stimulation unit comprising two first stimulus medium, wherein the two first stimulus medium are respectively connected to the top side part to correspond to F3 position and F4 position among the electrode positions of the International 10-20 EEG system;
a control unit electrically connected with the display headset, the speaker and the first stimulus medium, wherein the control unit stores programmed data information of the binaural beats music and the virtual image.

2. The training device for lowering intraocular pressure according to claim 1, wherein the first stimulus medium is a first magnetic stimulation medium, the first magnetic stimulation medium comprises an extension frame, a mounting seat and a magnetic coil, the extension frame is connected to the top side part, the mounting seat has a first end and a second end which are opposite, the first end is connected to the extension frame, and the magnetic coil is connected to the mounting seat and electrically connected with the control unit.

3. The training device for lowering intraocular pressure according to claim 2, wherein the mounting seat comprises a plurality of support layers stacked in sequence along the direction from the first end to the second end, and each support layer is provided with the magnetic coil, among any two adjacent support layers, the outer contour of one support layer close to the first end is larger than that of one support layer far from the first end, and the one support layer far from the first end is arranged in the middle of one support layer close to the first end.

4. The training device for lowering intraocular pressure according to claim 3, wherein an end face of the support layer far from the first end is a first magnetic surface, and the magnetic coil surrounds a first area around the center of the first magnetic surface on the first magnetic surface, among any two adjacent support layers, one support layer far from the first end is arranged in the first area of one support layer close to the first end.

5. The training device for lowering intraocular pressure according to claim 2, wherein the control unit can control the magnetic coil to emit a plurality of current waves within a predetermined time t, each current wave has a rated current value, and the rated current values of the current waves within the predetermined time t are gradually increasing from small to large, and the rated current values are between 1 A-10 A.

6. The training device for lowering intraocular pressure according to claim 1, wherein the first stimulus medium is a first electrical stimulation medium, which comprises a connecting seat and a conductive post, the first electrical stimulation medium has a third end and a fourth end which are opposite, the third end is connected to the bracket unit, and the conductive column is connected to the fourth end and electrically connected to the control unit; the number of the conductive columns is multiple, and the conductive columns are distributed on the end face of the fourth end in parallel; the conductive column comprises a connecting part and a silicon electrode part, the connecting part is provided with a fifth end and a sixth end which are opposite, the fifth end is connected to the fourth end, and the silicon electrode part is connected to the sixth end; the number of the silicon electrode parts is multiple, and the silicon electrode parts are distributed on the end face of the sixth end in parallel.

7. The training device for lowering intraocular pressure according to claim 1, wherein the transcranial stimulation unit further comprises two second stimulus medium, which are electrically connected with the control unit, and the two second stimulus medium are connected to the top side part to respectively correspond to the F7-T3 position, F8-T4 position of the user among the electrode positions of the International 10-20 EEG system.

8. The training device for lowering intraocular pressure according to claim 1, characterized by further comprising an eye mask unit and an head laser acupoint stimulation unit, wherein the eye mask unit comprises an eye mask body, two eyes surrounding heating parts and eyes surrounding acupoint stimulation parts; the eye mask body is detachably connected to the bracket unit and straddles the eyes of the user forward; the eye mask body is provided with eye holes for exposing the eyes of the user; and the eyes surrounding heating parts are electrically connected with the control unit. The two eyes surrounding heating parts is connected to the eye mask body, the eyes surrounding acupoint stimulation parts is electrically connected to the control unit, and eyes surrounding acupoint stimulation parts are connected to the eye mask body to respectively correspond to two Sibai ST2 acupoints, two Cuanzhu BL2 acupoints and two Qiuhou Ex-HN7 acupoints of the user; the head laser acupoint stimulation unit comprises two head laser acupoint stimulation media, two foot sleeves and four foot laser acupoint stimulation media, wherein the head laser acupoint stimulation media and the foot laser acupoint stimulation media are both electrically connected with the control unit; the two head laser acupoint stimulation media are connected to the back side part to respectively correspond to the two Fengchi GB20 acupoints of the user; the two foot sleeves are respectively worn by the feet of the user; and each foot sleeve is provided with two foot laser acupoint stimulation media to stimulate the user's two Pucan BL61 acupoints and two Shenmai BL62 acupoints.

9. The training device for lowering intraocular pressure according to claim 8, wherein the head laser acupoints stimulation medium and the foot laser acupoints stimulation medium are used for outputting laser with a wavelength of 650 nm-808 nm, and the flashing picture of the light frequency stimulation mode is green light with a flashing rate of 5 Hz-50 Hz.

10. The training device for lowering intraocular pressure according to claim 1, wherein the training device for lowering intraocular pressure further comprises an exercise bike unit, the exercise bike unit comprises a bike frame, a flywheel, a pedal group power module and an energy conversion module, the bike frame is used for the user to ride, the flywheel is rotatably connected to the bike frame, the pedal group is in transmission connection with the flywheel and is used for the user to step on to make the flywheel rotate around the central axis of the exercise bike, the energy conversion module is respectively connected with the exercise bike and the power module, and is used for converting the rotational potential energy of the exercise bike into electric energy and storing the electric energy in the power module, and the power module is electrically connected with the control unit.
